(19) United States
(12) Patent Application Publication
(10) Pub. No.: US 2014/0066786 A1
(43) Pub. Date: Mar. 6, 2014

(54) Method and Apparatus For Improved Wound Healing and Enhancement of Rehabilitation

(71) Applicants: Morteza Naghavi, Houston, TX (US); Albert A. Yen, Pearland, TX (US); Haider Ali Hassan, Houston, TX (US); David S. Panthagani, Houston, TX (US)

(72) Inventors: Morteza Naghavi, Houston, TX (US); Albert A. Yen, Pearland, TX (US); Haider Ali Hassan, Houston, TX (US); David S. Panthagani, Houston, TX (US)

(21) Appl. No.: 13/952,315

(22) Filed: Jul. 26, 2013

Related U.S. Application Data
(60) Provisional application No. 61/696,449, filed on Sep. 4, 2012.

(51) Int. Cl. A61B 17/135; A61B 5/026; A61B 5/024; A61B 5/145; A61B 5/00

(52) U.S. Cl. CPC A61B 17/1355; A61B 5/14542; A61B 5/4866; A61B 5/024; A61B 5/026; A61B 5/4836
USPC 600/481; 606/201; 606/202

(57) ABSTRACT

Methods and a device for improving wound healing and for improving the effects of rehabilitative therapies in patients with cognitive and motor deficits are provided. Repeated regimens of remote ischemic conditioning are performed. Markers of ischemia are monitored in the tissue. The remote ischemic conditioning regimen may be adjusted based on the monitoring results. The remote ischemic conditioning regimen can be performed at a hospital, medical clinic, healthcare facility, or at a subject's home.

### CROSS REFERENCE TO RELATED APPLICATIONS

This application claims priority based on U.S. Provisional Application No. 60/923,821 filed May 23, 2007, U.S. Provisional Application No. 60/969,863 filed Sep. 4, 2007, U.S. Provisional Application No. 61/025,715 filed Feb. 1, 2008, U.S. Provisional Application No. 61/029,147 filed Feb. 15, 2008, Patent Cooperation Treaty Provisional Application No. PCT/US08/64767 filed May 23, 2008, United States Utility Application U.S. Ser. No. 12/601,509 filed Nov. 23, 2009, and U.S. Application U.S. Ser. No. 12/323,392 filed on Nov. 25, 2008, U.S. Provisional Application 61/676,449 filed Jul. 27, 2012, the disclosures of which are incorporated herein by reference in their entireties.

### BACKGROUND

The disclosures herein relate generally to noninvasive ischemic conditioning treatment based on monitoring of elicited tissue ischemia and more particularly to methods and device to improve healing of acute and chronic wounds and to enhance or accelerate the effects of rehabilitative therapies in individuals with physical limitations.

Brief periods of ischemia (a local shortage of oxygen-carrying blood supply) in biological tissue are known in some systems to render that tissue more resistant to subsequent ischemic insults. This phenomenon is called ischemic conditioning, or, more specifically, ischemic preconditioning (prefix pre- for ‘before’). When the brief periods of ischemia are elicited in a tissue distant from the tissue that is rendered resistant to subsequent insults, the treatment is termed, ‘remote’ ischemic conditioning.

Further, for an organ or tissue already undergoing total or subtotal ischemia, brief periods of ischemia in a distant tissue in the same body has been shown to elicit a tissue protective effect in the original organ or tissue. This phenomenon has been termed, ischemic perconditioning (prefix per- for ‘during’).

Further, for an organ or tissue already undergoing total or subtotal ischemia, blood flow conditions can be modified during the onset of resumed blood flow to significantly reduce reperfusion injury. Since this method begins at the onset of resuming blood flow after ischemia, it is known as postconditioning (prefix post- for ‘after’).

Ischemic conditioning elicits tissue protection and appears to be a ubiquitous endogenous protective mechanism at the cellular level that has been observed in the heart of humans and other animal species tested. This protection has also been seen in organs such as the stomach, liver, kidney, gut, skeletal tissue, urinary bladder and brain. See D M Yellon and J M Downey, “Preconditioning the myocardium: from cellular physiology to clinical cardiology,” Physiol Rev 83 (2003) 1113-1151.

A standard ischemic preconditioning (IPC) stimulus of one or more brief episodes of non-lethal ischemia and reperfusion elicits a bi-phasic pattern of tissue protection. The first phase manifests almost immediately following the IPC stimulus and lasts for 1-2 h, after which its effect disappears (termed classical or early IPC). The second phase of tissue protection appears 12-24 h later and lasts for 48-72 h (termed the Second Window of Protection [SWOP] or delayed or late IPC). See D J Hausenloy and D M Yellon, “The Second Window of Preconditioning (SWOP) Where Are We Now?” Cardiovasc Drugs Ther 24 (2010) 235-254.

The inventors have previously taught that additive biochemical, physiological, and tissue protective effects may be observed by performing repeated ischemic conditioning regimens. Additive effects have previously been termed “stacking” by the inventors. See Patent Cooperation Treaty Provisional Application No. PCT/US08/64767 filed May 23, 2008, United States Utility Application U.S. Ser. No. 12/601,509 filed Nov. 23, 2009, and U.S. Application U.S. Ser. No. 12/323,392 filed on Nov. 25, 2008.

Wound healing, or wound repair, is the body's natural process of regenerating tissue. When an individual is wounded, a set of complex biochemical events takes place to repair the damage. However, this process is not only complex but fragile, and susceptible to interruption or failure leading to the formation of chronic non-healing wounds. Chronic wounds are defined as wounds, which have failed to proceed through an orderly and timely reparative process to produce anatomic and functional integrity over a period of 3 months. Factors which may contribute to delayed wound healing and the development of chronic wounds include diabetes, venous or arterial disease, old age, and infection. Chronic wounds often display a pro-inflammatory phenotype with poor vascularity.

Stroke and traumatic brain injury (TBI) are common, serious, and disabling global health care problems. Rehabilitation is a major part of patient care of these and other health conditions which often result in prolonged period of reduced mobility and limited physical activity. Although patient outcome is heterogeneous and individual recovery patterns differ, several studies suggest that recovery of body functions and activities is predictable in the first days after stroke. See P Langhorne, J Bernhardt, and G Kwakkel, “Stroke Rehabilitation,” Lancet 377 (2011) 1693-1702.

For patients with stroke or TBI, the rate of spontaneous neurological recovery is often highest during the first days or weeks following the initial brain insult. Once recovery slows, it seldom accelerates again. Thus, the optimal time for instillation of an aggressive rehabilitative therapy plan is during this initial time period, but many post-stroke and post-TBI patients have disabling motor and cognitive deficits and are unable to participate fully and benefit from intensive rehabilitative therapies.

What are needed are device and methods that adapt pre-, per-, post-, and repeated ischemic conditioning treatments to novel clinical applications in the areas of wound healing and rehabilitation.

### SUMMARY

Provided herein are methods and apparatus for ischemic conditioning to reduce damage to tissues and/or improve response to therapies. In one embodiment, ischemic conditioning is effected by transiently and repeatedly administering transient ischemia to at least one vascular area of a patient or part thereof. In an embodiment, protective and/or therapeutic effects of ischemic conditioning can be enhanced by adjusting duration and frequency of ischemic conditioning protocols over a period of time. In an embodiment, effects of ischemic conditioning can be enhanced by administering multiple ischemic conditioning protocols over a period of time.

In an embodiment, an ischemic conditioning protocol can be specifically adapted to provide both early and delayed protective effects. In an embodiment, an ischemic conditioning protocol is adapted for occlusion of capillaries based on external pressure. In an embodiment, an ischemic conditioning protocol is implemented by a programmable device that is capable of tissue ischemia monitoring. In an embodiment, monitoring of oxygenation and metabolic markers of tissue ischemia is provided simultaneously with ischemic conditioning. In an embodiment, an ischemic conditioning protocol is adjusted based on monitoring of desired tissue markers, including but not limited to tissue ischemia markers of oxygenation and metabolism. Alternatively, the invention is provided with only monitoring of pulse or blood flow instead of ischemia, or without monitoring altogether, to improve ease of use.

The protective and therapeutic effects conferred by ischemic conditioning can be systemic or local to the ischemic tissue. In an embodiment, the ischemic conditioning can is administered to the location of an anticipated tissue injury. In another embodiment, the ischemic conditioning is administered after a medical intervention, such as a surgical procedure or the creation of a wound.

In an embodiment, a device for ischemic conditioning is provided. In one embodiment the device has one or more occluding members in addition to programmable controlling members and/or data storage members. A sensor for monitoring of tissue markers may be additionally provided. The occluding member may be adapted to at least partially occlude an internal vascular lumen to reduce or occlude flow to at least one peripheral tissue of the patient. In an embodiment, external skin pressure is provided to induce ischemia only at the skin and/or subdermal levels. The programmable controlling member can be adapted to control the frequency and duration of ischemia in a tissue according to an ischemic conditioning protocol. In an embodiment, the programmable controlling member is programmed by a separate device. The data storage member, such as a computer, can store the protocol and/or monitoring results. An optional display may be provided to show the ischemic conditioning protocol, stored data, results of the ischemic conditioning, and/or other relevant data. The devices as described herein may adapted for home or clinical use. For example, a device for home use may simply utilize external cuff occlusions around an extremity, blood pressure measurement, and/or pulse monitoring.

In one embodiment wherein the vascular conditioning treatment includes induced ischemia the induced ischemia is sufficient to induce reactive hyperemia in the distal extremity including both hands, both feet, both hands and feet, and/or portions thereof. The method may be complemented by instructing a schedule of hand exercises to the patient.

In one embodiment employing induced ischemia as a preconditioning treatment, the induced ischemia is transiently and repeatedly induced in at least one limb or portion thereof of a patient according to a schedule of vascular occlusions prior to initiating the intervention in the patient. Alternatively or in addition to other preconditioning treatments, in one embodiment heat sufficient to induce vasodilatation is applied to at least one distal extremity of the patient. The heat may be generated by electric heating, ultrasound, microwave (MW), photo thermal energy, infrared (IR), radio frequency (RF) energy or heat derived from chemical reactions such as oxidation.

In other embodiments of the invention, apparatus for transiently and repeatedly inducing heat in a peripheral vascular area of a patient is provided that includes use of a plurality of heating elements such that both hands and/or feet are transiently heated. The apparatus may be manual in operation or may be automated. In one embodiment the apparatus includes a programmable monitor for instructing heating in accordance with a schedule.

### BRIEF DESCRIPTION OF THE DRAWINGS

FIG. 1 depicts several locations for placement of noninvasive cuffs for inducing ischemic conditioning.

FIG. 2A depicts an embodiment of inflatable cuffs that can be curved when flat and closed by fasteners to be conical and/or adjustable. FIG. 2B depicts an embodiment of inflatable thigh cuffs that are secured to a molding.

FIG. 3A depicts an ambulatory embodiment enabling the patient to wear one or more noninvasive cuffs together with a controlling unit for scheduled inflation of the noninvasive cuff(s). FIG. 3B depicts two locations for placement of cuffs on arms. FIG. 3C depicts an embodiment of the placement of cuffs on two arms and two legs for ischemic conditioning.

FIG. 4A depicts a schematic of an example of early, or acute, and delayed SWOP therapeutic effects to be expected upon a single administration of ischemic conditioning. FIG. 4B depicts a schematic of an example of a “stacking” effect that is expected to result from performing two or more regimens of ischemic conditioning.

FIG. 5A depicts an embodiment of a device that is implemented for home use of ischemic conditioning. FIG. 5B depicts another embodiment of a device that is implemented for home use of ischemic conditioning. FIG. 5C depicts an embodiment of a device that is implemented for home use which can also be calibrated or programmed by a separate device that is capable of tissue ischemia monitoring.

FIG. 6 depicts a system for ischemic conditioning.

FIG. 7 depicts an example of thresholds of ischemic effect on a tissue with which an ischemic conditioning protocol can be adjusted to prevent or reduce tissue injury.

FIG. 8A depicts cross sectional views of an embodiment of applying superficial pressure around an extremity. FIGS. 8B-D depict cross sectional views of embodiments of superficial pressure as applied against a body surface such as the skin.

FIG. 9A depicts placement of implementations that be adapted for treatments including inflation to drive blood from surface tissues and heating to induce vasodilation, both a prophylaxis. FIG. 9B depicts a glove implementation wherein each finger is isolated. FIGS. 9C and 9D depict cap implementations.

FIGS. 10-14 depict other embodiments for inflatable compression of the arm and hand for ischemic conditioning.

FIG. 15 depicts an embodiment of a pressured body suit that delivers external pressure to create ischemia at the skin and subdermal tissue levels.

FIGS. 16A-B depict embodiments of a mattress capable of preventing or reducing bedsores by ischemic conditioning.

FIGS. 17A-F show data indicating variations in tissue oxygenation between individuals.

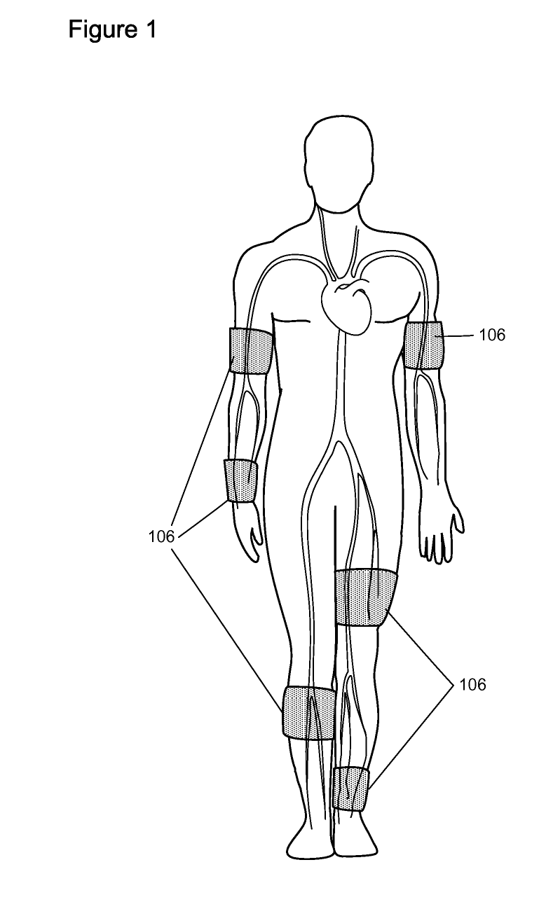

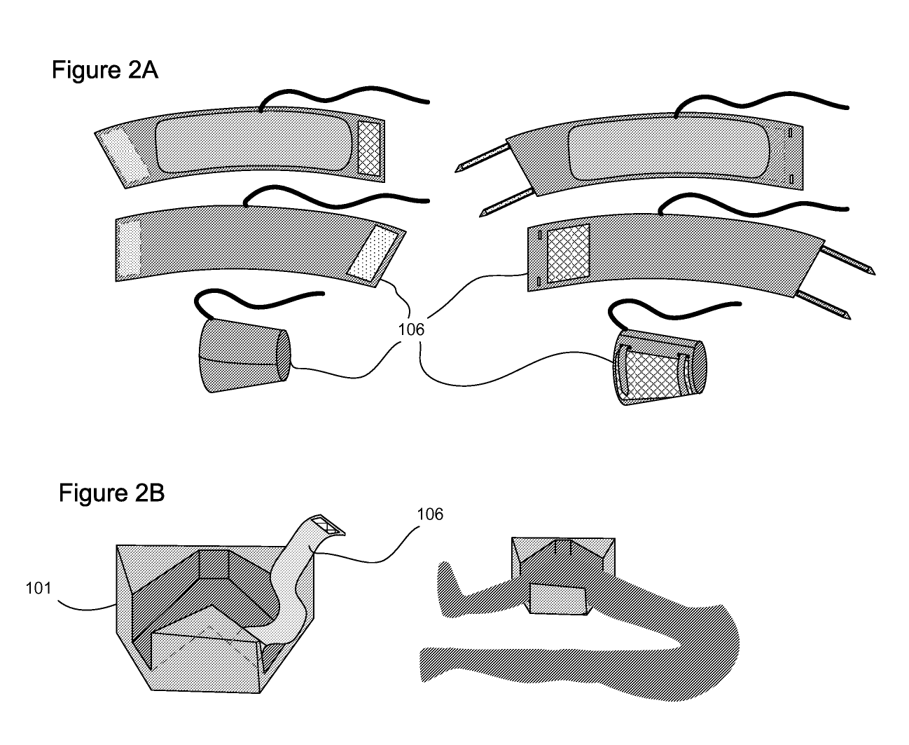

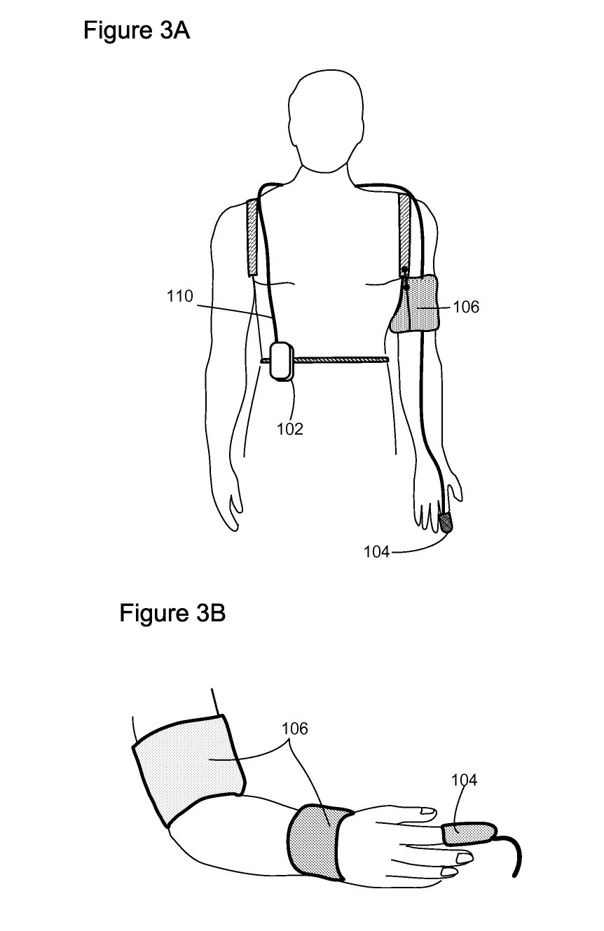

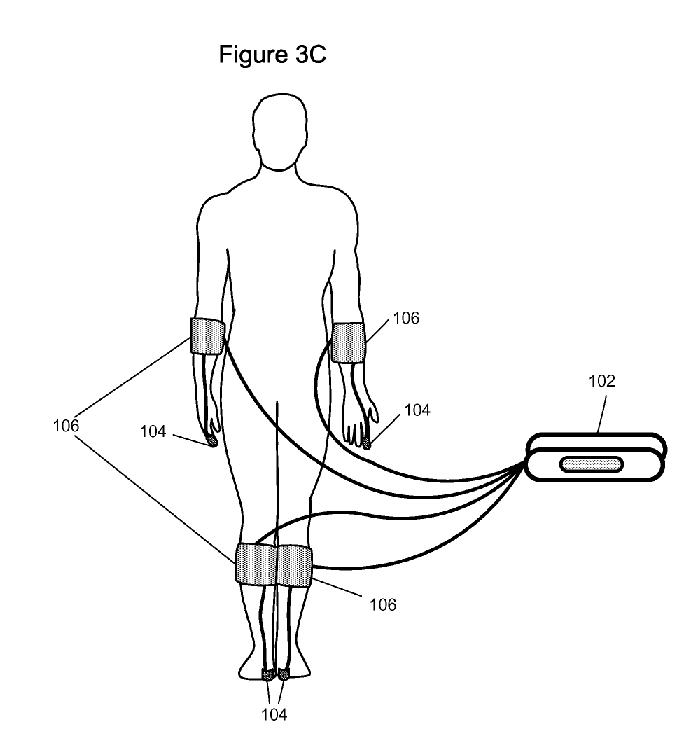

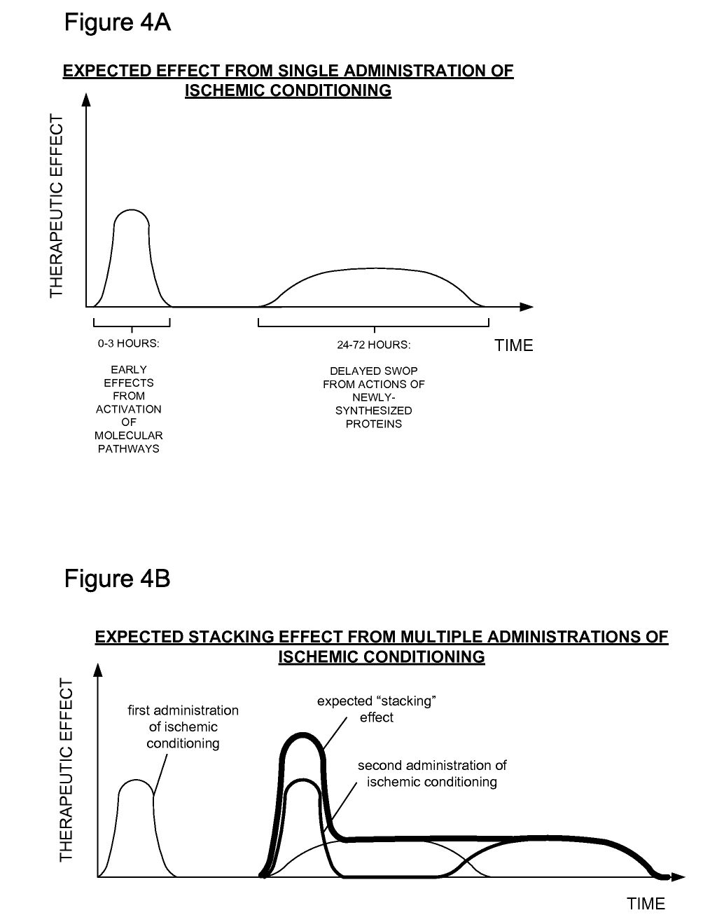

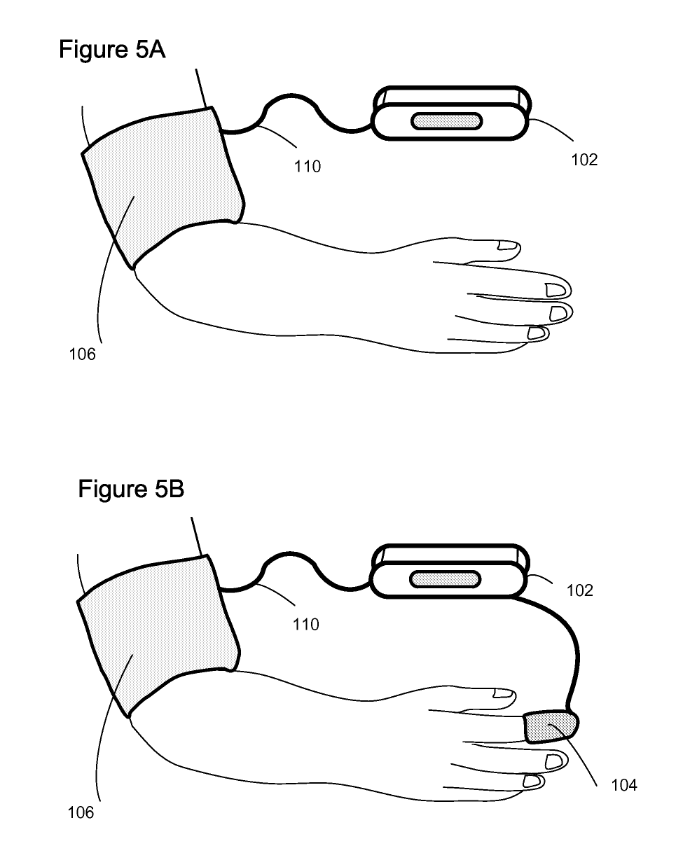

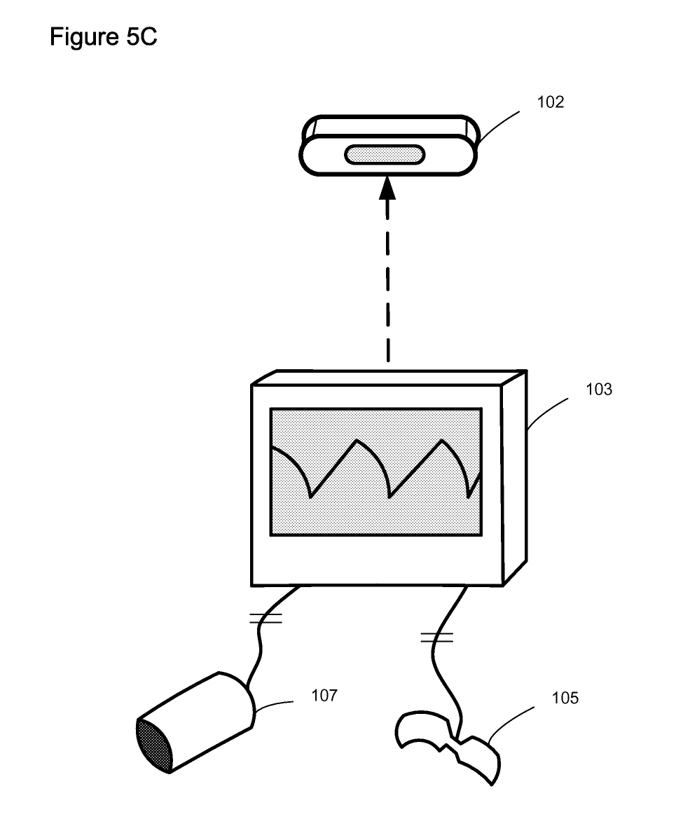

### DETAILED DESCRIPTION

Without limiting the scope of the invention, the invention is described in connection with ischemic preconditioning, perconditioning, and postconditioning of natural properties of tissues for improving the rate of healing acute and chronic wounds and for enhancing the beneficial effects of rehabilitative therapies in individuals with cognitive and/or motor deficits. Ischemic preconditioning is a remarkable phenomenon within medical science. Eliciting brief periods of ischemia (a local shortage of oxygen-carrying blood supply) in biological tissue will render that tissue more resistant to subsequent ischemic insults. This method is known as preconditioning. Further, for an organ or tissue already undergoing total or subtotal ischemia, blood flow conditions can be modified during the onset of resumed blood flow to significantly reduce reperfusion injury. Since this method begins at the onset of resuming blood flow after ischemia, it is known as postconditioning.

The present inventors have adapted the experimental phenomena of ischemic conditioning to useful preventative and therapeutic measures for a myriad of novel indications. In certain embodiments, the process is monitored and controlled as well as individualized the physiology of individual patients. The controlled induced ischemia disclosed and implemented herein provides conditioning to increase effects of therapies and decrease the incidence and extent of tissue injury by several mechanisms, e.g. increased scavenging of free radicals induced by trauma and reduction in inflammation. In other embodiments, the administration of controlled induced ischemia is adapted to increase functional capillary density in desired sites with an outcome of hastened wound healing. As used herein the term “ischemia” means lowering of baseline blood flow to a tissue. The term “hypoxia” means lowering of arterial PO2. Both ischemia and hypoxia in distal extremities can be induced by partial or complete occlusion of blood supply upstream of the extremity. By “distal extremity” it is meant the hands and feet, including the digits of the hands and feet. By “regional or local” it is meant administration to a defined area of the body as contrasted with systemic administration. In an embodiment the occlusion is sufficient to induce reactive hyperemia in at least one limb or portion thereof. “Reactive hyperemia” is a term that can be defined as an increase in blood flow to an area that occurs following a brief period of ischemia (e.g., arterial occlusion). One embodiment of the present invention employs controlled administrations of ischemia to condition tissues of target areas. By “target areas” it is meant areas known to exhibit injury expected to tissues during medical, surgical and other pharmacological interventions or non-pharmacological injuries. The term “ischemic conditioning” means inducing one or more episodes of ischemia that are controlled by monitoring of one or more biochemical markers in a target area.

### Ischemic Preconditioning

The benefits of ischemic preconditioning have been observed in myocardial tissue of dogs that were pretreated by alternately manually clamping and unclamping coronary arteries to intermittently turn off the blood flow to the heart. Dogs who were treated with an optimal number of four cycles of five-minute coronary occlusion followed by five-minute reperfusion, exhibited 75% smaller infarct sizes resulting from a subsequent forty-minute coronary occlusion. Fewer than four cycles of coronary occlusion resulted in insufficient preconditioning in the dog model. Myocardial tolerance to injury also develops in response to treatment that does not include coronary occlusion (i.e., ischemia) but otherwise increases demand for oxygenated blood. In dogs, a treatment comprising of five five-minute periods of tachycardia alternating with five minutes of recovery has also been shown to reduce infarct sizes.

The myocardial resistance to infarct resulting from brief periods of ischemia has been described in other animal species including rabbit, rat and pig. Ischemic preconditioning has also been demonstrated in humans. A second coronary occlusion during the course of coronary angioplasty often results in less myocardial damage than the first. Naturally occurring ischemic preconditioning of the myocardium has been found in humans suffering from bouts of angina.

Ischemic preconditioning occurs not only in myocardial tissue but also occurs in non-cardiac tissue including kidney, brain, skeletal-muscle, lung, liver and skeletal tissue. Further, resistance to infarct exists even in virgin tissue following brief ischemia in spatially remote cardiac or non-cardiac tissue. Ischemic preconditioning also exhibits a temporal reach: an early phase develops immediately within minutes of the preconditioning ischemic injury and lasts for a few hours, and a late phase develops approximately twenty four hours later and can last for several days.

### Perconditioning

In addition to preconditioning for reducing damage resulting from an anticipated injury, ischemic conditioning treatments can be performed during an acute ischemic event, such as acute coronary syndrome, transient cerebrovascular ischemic attack, or stroke. This is known as ischemic perconditioning. Schmidt et al. (Am J Physiol Heart Circ Physiol, 292: H1883-H1890, 2007) have shown the effectiveness of this approach in reducing myocardial injury in pigs. More recently, Botker et al. (“Prehospital remoteischemic perconditioning reduces infarct size in patients with evolving myocardial infarction undergoing primary percutaneous intervention.” 58th Annual Scientific Sessions of American College of Cardiology, Orlando; March, 2009) reported the beneficial effects of this approach in human patients with acute coronary syndrome; however, those researchers did not utilize ischemia monitoring during ischemic conditioning treatments. The inventors believe that performing ischemic conditioning treatments without ischemia monitoring does not guarantee that the treatments have been properly performed. Ischemic conditioning treatments performed with ischemia monitoring, as described in this patent application, can provide an accurate and operator-independent platform for adoption of ischemic conditioning in patient care.

### Postconditioning

Timely reperfusion to reduce the duration of ischemia is the definitive treatment to prevent cellular injury and necrosis in an ischemic organ or tissue. However, defined as reperfusion injury, additional damage can occur to an organ by the uncontrolled resumption of blood flow after an episode of prolonged ischemia. This damage is distinct from the injury resulting from the ischemia per se. One hallmark of reperfusion injury is that it may be attenuated by interventions initiated before or during the reperfusion. Reperfusion injury results from several complex and interdependent mechanisms that involve the production of reactive oxygen species, endothelial cell dysfunction, microvascular injury, alterations in intracellular Ca2+ handling, changes in myocardial metabolism, and activation of neutrophils, platelets, cytokines and the complement system. Deleterious consequences associated with reperfusion include a spectrum of reperfusion-associated pathologies that are collectively called reperfusion injury. Reperfusion injury can extend not only acutely, but also over several days following a medical or surgical intervention.

For example, even with successful treatment of occluded vessels, a significant risk of additional tissue injury after reperfusion may still occur. Typically, reperfusion after a short episode of myocardial ischemia is followed by the rapid restoration of cellular metabolism and function. However, if the ischemic episode has been of sufficient severity and/or duration to cause significant changes in the metabolism and the structural integrity of tissue, reperfusion may paradoxically result in a worsening of function, out of proportion to the amount of dysfunction expected simply as a result of the duration of blocked flow. Although the beneficial effects of early reperfusion of ischemic myocardium with thrombolytic therapy, PTCA, or CABG are now well established, an increasing body of evidence indicates that reperfusion also induces an additional injury to ischemic heart muscle, such as the extension of myocardial necrosis, i.e., extended infarct size and impaired contractile function and metabolism. Hearts undergoing reperfusion after transplantation also undergo similar reperfusion injury events. Similar mechanisms of injury are observed in all organs and tissues that are subjected to ischemia and reperfusion.

Thus, in general, all organs undergoing reperfusion are vulnerable to reperfusion injury. Postconditioning is a method of treatment for significantly reducing reperfusion injury to an organ or tissue already undergoing total or subtotal ischemia. Postconditioning involves a series of brief, iterative interruptions in arterial reperfusion applied at the immediate onset of reperfusion. The bursts of reflow and subsequent occlusive interruptions last for a matter of seconds, ranging from at least around 60 second intervals in larger animal models to 5-10 second intervals in smaller rodent models. Preliminary studies in humans used 1 minute intervals of reperfusion and subsequent interruptions in blood flow during catheter-based percutaneous coronary intervention (PCI).

The spatial and temporal characteristics of ischemic preconditioning and postconditioning may be a manifestation of complex interactions between various underlying phenomena. The numerous biochemical and cellular mechanisms underlying the phenomena of ischemic conditioning are still being researched and are not fully understood. These research efforts have been motivated at least in part by the hope of developing pharmaceutical drugs which would provide the infarct sparing effect of ischemic conditioning.

### Ischemic Conditioning Protection at the Cellular and Biochemical Level

Ischemia has been shown to produce tolerance to damage from subsequent ischemic damage. Ischemia preconditioning was first described by Murry et al who found that protection was conferred to ischemic myocardium by preceding brief periods of sublethal ischemia separated by periods of reperfusion. (Murry C E, Jennings R B, Reimer K A. Circulation 74(5) (1986) 1124-36). As a consequence of four five-minute episodes of regional ischemia in the canine myocardium, a net effect of 75 percent reduction in infarct size compared to a control group.

The protective effects of conditioning may be mediated by signal transduction changes to tissues. The current paradigm suggests that nonlethal episodes of ischemia reduce infarct size. Ischemic conditioning has been found to lead to the release of certain substances, such as adenosine and bradykinin. These substances bind to their G-protein-coupled receptors and activate kinase signal transduction cascades. See Id. These kinases converge on the mitochondria, resulting in the opening of the ATP-dependent mitochondrial potassium channel. See Garlid K D et al. “Cardioprotective effect of diazoxide and its interaction with mitochondrial ATP-sensitive K⁺ channels. Possible mechanism of cardioprotection.” Circ Res 81 (1997) 1072-1082. Reactive oxygen species are then released. See Vanden Hoek T L et al., “Reactive oxygen species released from mitochondria during brief hypoxia induce preconditioning in cardiomyocytes.” J Biol Chem 273 (1998) 18092-18098. Thus additional protective signaling kinases can be activated, such as heat shock inducing protein kinase C.

Further, the signaling kinases mediate the transcription of protective distal mediators and effectors, such as inducible nitric oxide synthase, manganese superoxide dismutase, heat-stress proteins and cyclo-oxygenase 2, which manifest 24-72 hours after infarction to provide late protection. Suggested mechanisms of how these signaling transduction pathways mediate protection and ultimately reduce infarct size include maintenance of mitochondrial ATP generation, reduced mitochondrial calcium accumulation, reduced generation of oxidative stress, attenuated apoptotic signaling and inhibition of mitochondrial permeability transition-pore (mPTP) opening. See D M Yellon and J M Downey, “Preconditioning the myocardium: from cellular physiology to clinical cardiology,” Physiol Rev 83 (2003) 1113-1151; Yellon D M, Hausenloy D J, “Realizing the clinical potential of ischemic preconditioning and postconditioning,” Nat Clin Pract Cardiovasc Med. 2(11)(2005) 568-75. It is also possible that alternative protective mechanisms of ischemic conditioning might exist that are independent of signal transduction pathways, such as those mediated by antioxidant and anti-inflammatory mechanisms, and so on.

Even further, formation of vascular collaterals is also induced by ischemia and hypoxia of blood vessels. Vascular endothelial growth factor (VEGF) production can be induced in cells that are not receiving enough oxygen. When a cell is deficient in oxygen, it produces the transcription factor Hypoxia Inducible Factor (HIF). HIF stimulates the release of VEGF among other functions including modulation of erythropoeisis. Circulating VEGF then binds to VEGF receptors on endothelial cells and triggers a tyrosine kinase pathway leading to angiogenesis.

Ischemia has been shown to produce tolerance to reperfusion damage from subsequent ischemic damage. One physiologic reaction to local ischemia in normal individuals is reactive hyperemia to the previously ischemic tissue. Arterial occlusion results in lack of oxygen (hypoxia) as well as an increase in vasoactive metabolites (including adenosine and prostaglandins) in the tissues downstream from the occlusion. Reduction in oxygen tension in the vascular smooth muscle cells surrounding the arterioles causes relaxation and dilation of the arterioles and thereby decreases vascular resistance. When the occlusion is released, blood flow is normally elevated as a consequence of the reduced vascular resistance.

Perfusion of downstream tissues is further augmented by flow-mediated dilation (FMD) of larger conduit arteries, which acts to prolong the period of increased blood flow. As a consequence of the elevated blood flow induced by reactive hyperemia, downstream conduit vessels undergo luminal shear stress. Endothelial cells lining the arteries are sensitive to shear stress and the stress induces in opening of calcium-activated potassium channels and hyperpolarization of the endothelial cells with resulting calcium entry into the endothelial cells, which then activates endothelial nitric oxide synthase (eNOS). Consequent nitric oxide (NO) elaboration results in vasodilation. Endothelium-derived hyperpolarizing factor (EDHF), which is synthesized by cytochrome epoxygenases and acts through calcium-activated potassium channels, has also been implicated in flow-mediated dilation. Endothelium derived prostaglandins are also thought to be involved in flow-mediated dilation.

Ischemia preconditioning has been found to have remote and systemic protective effects in both human and animal models. Transient limb ischemia (3 cycles of ischemia induced by cuff inflation and deflation) on a contralateral arm provides protection against ischemia-reperfusion (inflation of a 12-cm-wide blood pressure cuff around the upper arm to a pressure of 200 mm Hg for 20 minutes) induced endothelial dysfunction in humans and reduces the extent of myocardial infarction in experimental animals (four cycles of 5 minutes occlusion followed by 5 minutes rest, immediately before occlusion of the left anterior descending (LAD) artery). (Kharbanda R K, et al. Circulation 106 (2002) 2881-2883.)

Recent evidence in a skeletal muscle model has suggested that IPC results in increased functional capillary density, prevention of ischemia/reperfusion induced increases in leukocyte rolling, adhesion, and migration, as well as upregulation of expression of nNOS, iNOS, and eNOS mRNA in ischemia reperfusion injured tissue. (Huang S S, Wei F C, Hung L M. “Ischemic preconditioning attenuates postischemic leukocyte—endothelial cell interactions: role of nitric oxide and protein kinase C” Circulation Journal 70 (8) (2006) 1070-5). Research has also shown that ischemic preconditioning can result in elevations of heat shock proteins, antioxidant enzymes, Mn-superoxide dismutase and glutathione peroxidase, all of which provide protection from free radical damage. (Chen Y S et al. “Protection ‘outside the box’ (skeletal remote preconditioning) in rat model is triggered by free radical pathway” J. Surg. Res. 126 (1) (2005) 92-101).

Although originally described as conferring protection against myocardial damage, preconditioned tissues have been shown to result in ischemia tolerance through reduced energy requirements, altered energy metabolism, better electrolyte homeostasis and genetic re-organization, as well as reperfusion tolerance due to less reactive oxygen species and activated neutrophils released, reduced apoptosis and better microcirculatory perfusion compared to non-preconditioned tissue. (Pasupathy S and Homer-Vanniasinkam S. “Ischaemic preconditioning protects against ischaemia/reperfusion injury: emerging concepts” Eur. J. Vasc. Endovasc. Surg. 29 (2) (2005) 106-15).

### Ischemic Conditioning Based on Monitoring of Tissue Markers

In accordance with the novel indication of the present invention, in an embodiment the body's own adaptive responses to induced ischemia or hypoxia are monitored to provide protection against tissue damage and to increase response to therapies. In an embodiment of the invention, duration and frequency of ischemia are adjusted based on monitoring of markers in a target tissue, including but not limited to metabolic, oxygenation, and/or biochemical markers. In an embodiment, supplemental episodes of heat, vibration, drugs, or combinations thereof, are provided based on monitoring of biochemical markers in the target tissue.

Several studies have indicated that there may be organ-specific biochemical thresholds for dysoxia, and yet heterogeneity of blood flow (or cellular metabolism) within an organ can also lead to different values at different locations within the same organ. For example, for a discussion of pH thresholds related to hepatic dysoxia, see, inter alia, Soller B R et al. “Application of fiberoptic sensors for the study of hepatic dysoxia in swine hemorrhagic shock.” Crit Care Med. 2001 July; 29(7):1438-44. Further, overall tissue oxygen sufficiency can be confirmed by near-infrared measurement of cytochrome oxidase and the redox behavior of cytochrome oxidase during an operation is a good predictor of postoperative cerebral outcome. (Kakihana Y, et al., “Redox behavior of cytochrome oxidase and neurological prognosis in 66 patients who underwent thoracic aortic surgery.” Eur J Cardiothorac Surg. 2002 March; 21(3):434-9.)

Accordingly, chronic, regular or periodic administration of ischemia can be optimized to suit the variable needs of the target area prior to an injurious intervention. For example, the individual patient may schedule a pattern of ischemia, such as for limited periods 5-10 times a day for a period preceding each intervention. In another embodiment, ischemia is administered to the future injury site for a period prior to injury. Depending on responses desired and obtained in the individual patient, the intensity and duration of ischemia can be tuned for optimal responses.

Further, in an embodiment, sensing and monitoring of markers can provide measurements to control ischemic preconditioning and postconditioning. In an embodiment, the target tissue has been at least partially damaged prior to inducing ischemia. In an embodiment, ischemia is controlled by postconditioning at the onset of reperfusion to reduce reperfusion injury. In an embodiment, ischemic preconditioning reduces damage to tissue due to a traumatic medical procedure such as surgery, angioplasty, chemotherapy, or radiation. In an embodiment, ischemia and heat can also be similarly adjusted to increase monitored effects of certain therapies, such as drugs and radiotherapy. For example, in an embodiment, neuropathy from chemotherapy and radiotherapy interventions can be reduced or prevented by providing ischemic preconditioning based on monitoring levels of oxygen in a target tissue.

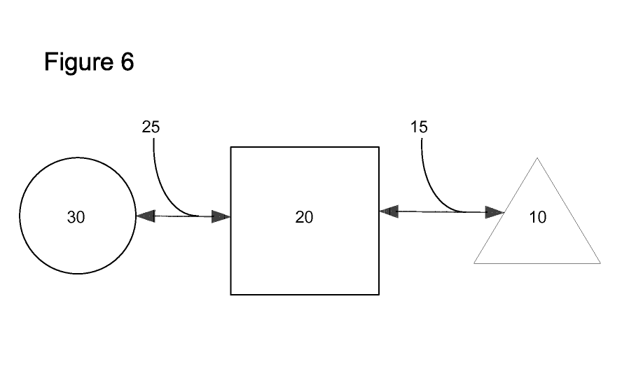

Ischemia can be controlled based on monitoring of biochemical markers by a system for ischemic conditioning. In an embodiment as depicted in FIG. 6, a system for ischemic conditioning can include an occluding device (10), a controlling device (20), a sensing device (30), and communication signals (15, 25) between the devices. The occluding device (10) induces ischemia through one or more episodes of occlusion of blood supply. The occluding device (10) is controllable by the controlling device (20) via a signal (15). The sensing device (30) is adapted to measure one or more biochemical markers in a target tissue and send information via a signal (25) to the controlling device (20). Accordingly, the controlling device (20) can control the one or more episodes of occlusion by the occluding device (10) based on monitoring of a signal (25) received from the sensing device (30).

Considering the occluding device in more detail, ischemia can be induced through one or more episodes of occlusion of blood supply by the occluding device. In an embodiment, the occluding device can be noninvasive. In an embodiment, the occluding device can induce occlusions at a duration and frequency suitable for the size of blood vessels and target tissue being conditioned. For example, in an embodiment, larger forearm arteries can be occluded at a longer duration and slower frequency than smaller blood vessels, such as those found in the fingers. In an embodiment, arterial occlusion is desirable in tissues with loose capillary walls as occlusion of the venous system in such tissues can result in unwanted leakage of plasma or blood into the tissue. However, in an another embodiment, to induce ischemia when arterial access for occlusion is unavailable, venous occlusion can be beneficial to prevent or reduce venous blood flow and in turn prevent or reduce arterial blood flow.

The duration and frequency of ischemia varies by therapeutic targets, but both duration and frequency of occlusions can be sustained for longer periods depending on the extent of occlusion. For example, within the same individual, the duration and frequency of ischemic conditioning can be adjusted to suit the faster metabolisms of tissues in the brain or heart as opposed to the slower metabolisms of other tissues, e.g. hair. Further, in an embodiment, the duration and frequency of ischemic conditioning can be adjusted to suit metabolic differences across individuals. Also, occlusion and release (reactive hyperemia) procedures with different durations and frequencies are implemented depending on individual tolerance and response to therapy. In an embodiment, duration and frequencies can vary upon a planned intervention schedule so that a desired distal and or contralateral vascular/neuro/neurovascular function is obtained. Occlusion and release is tailored to improve vasoreactivity (increasing the vasodilative capacity) by improving nitric oxide bioavailability (reducing destruction or increasing production). This effect can be seen in the same distal extremity as the occlusion but is also expected to have neurovascular mediated vasodilation of the contralateral extremity as well.

Considering the controlling device and sensing device in more detail, duration and frequency of ischemia and thermal conditioning can be adjusted by the controlling device based on monitoring of tissue markers of metabolic activity and/or therapeutic effects in the target tissue by the sensing device. For example, if levels of oxygen are monitored as dropping significantly into dysoxia and irreversible injury, the controlling device can alter ischemic episodes to decrease or stop until oxygen levels are monitored to be at a suitable range. Once reaching a desirable range, the ischemic episodes can resume under further monitoring. In an embodiment, a significant enough change in oxygen saturation levels to trigger a conditioning response can be at least 1%. In an embodiment, a significant enough change in oxygen saturation levels to trigger a conditioning response can vary depending on clinical conditions including areas of occlusion, areas of target tissue, duration and frequency of ischemia, and individual tolerance and response to therapy.

Similarly, if levels of other tissue markers of ischemia, including but not limited to lactate, pH, carbon dioxide, ATP, ADP, nitric oxide, peroxinitrate, electrolytes, free radicals, and combinations thereof, are determined to be changing significantly, the controlling device can adjust ischemic episodes until those levels are monitored to be at a suitable level again. Once reaching a desirable range, the ischemic episodes can resume under further monitoring. In an embodiment, a significant enough change in saturation levels of any marker to trigger a conditioning response can be at least 1%. In an embodiment, a significant enough change in saturation levels of markers to trigger a conditioning response can vary depending on clinical conditions including areas of occlusion, the particular target tissue, and duration and frequency of ischemia.

Further, if levels of other tissue markers of ischemic conditioning therapy, including but not limited to responses to chemotherapy, radiotherapy, neuropathy, hypertension, chronic conditions, operative outcome, and/or wound healing, are determined to be changing significantly, the controlling device can adjust ischemic episodes until those levels are monitored to be at a suitable level again. Once reaching a desirable range, the ischemic episodes can resume under further monitoring. For example, if tissue markers of chemotherapy induced neuropathy indicate an increase in tissue injury, the frequency of ischemic conditioning treatments can be decreased to prevent or reduce such injury. In an embodiment, measurement of tissue markers of response to ischemic conditioning treatments can include but are not limited to: adenosine, cytochrome oxidase, redox voltage, erythropoietin, bradykinin, opioids, ATP/ADP, and/or related receptors.

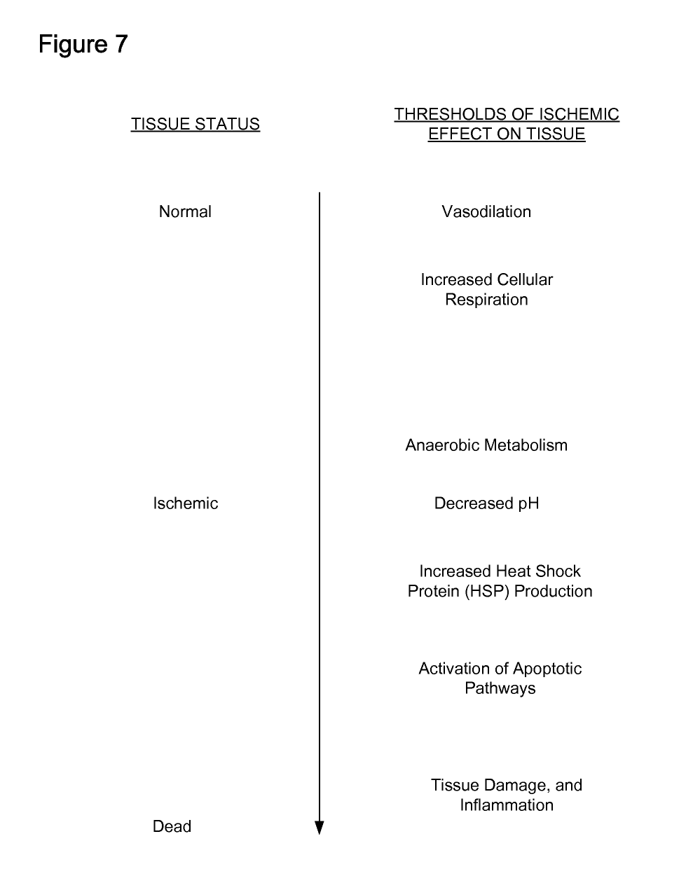

Monitoring can be continuous or intermittent, depending on the target tissues and the character of the intervention. For example, monitoring of tissues with slower inherent metabolic rate can be undertaken with more intermittent monitoring than those with high metabolic rates, such as cardiac tissue. Thus, in an embodiment, the desired frequency of monitoring of markers can depend on the extent of the induced ischemia and target tissue areas. In an embodiment, monitoring of tissue markers can provide data to satisfy thresholds of ischemia to adjust the ischemic conditioning protocol in order to prevent or minimize cell injury. For example, FIG. 7 depicts an example of thresholds of ischemic effect on a tissue with which an ischemic conditioning protocol can be adjusted to prevent or reduce tissue injury.

In an embodiment, biochemical markers in the target tissue include levels of lactate, pH, oxygen, carbon dioxide, ATP, ADP, nitric oxide, peroxinitrate, electrolytes, free radicals, and combinations thereof. In an embodiment, anaerobic conditions during ischemia can change levels of these biochemical markers of metabolic activity in the target tissue. For example, anaerobic respiration can cause lactate levels to increase, pH levels to decrease, oxygen levels to decrease, ATP levels to decrease, and ADP levels to increase. Other biochemical changes can also be measured in the target tissue, such as shifted levels of nitric oxide and peroxinitrate, electrolytes, and free radical redox states. Further, in an embodiment, the induced ischemia is modified and controlled until levels of the biochemical markers are measured to return to desirable ranges.

In an embodiment, biochemical marker measurement can also include thermal markers in the target tissue. Thermal markers can include levels of perfusion, carbon dioxide, external and inherent temperatures, and combinations thereof. Inherent skin temperature means the unaltered temperature of the skin. This is in contrast to an induced skin temperature measurement which measures perfusion by clearance or wash-out of heat induced on the skin. Various methods of recording of inherent skin temperature on a finger tip or palm distal to a noninvasive cuff are disclosed in Naghavi et al., U.S. application Ser. No. 11/563,676 and PCT/US2005/018437 (published as WO2005/118516). The combination of occlusive means and skin temperature monitoring has been termed Digital Temperature Monitoring (DTM) by the present inventor. In an embodiment, the method for monitoring the hyperemic response further includes simultaneously measuring and recording additional physiologic parameters including but not limited to pulse rate, blood pressure, galvanic response, sweating, core temperature, and/or skin temperature on a thoracic or truncal (abdominal) part.

In an embodiment, tissue markers can be measured noninvasively by suitable well known non-invasive probes in the art, such as, for example, the use of a pulse oximeter for measurement of oxygen saturation. In an embodiment, invasive measurement of biochemical markers can be performed by any suitable well known invasive probes in the art, such as, for example, fluorescent probes for nitric oxide measurement and sodium and potassium probes for electrolyte measurement. In an embodiment, invasive measurement of biochemical markers can include adapting a sensory mechanism together with a delivery catheter. In an embodiment, the tissue markers can be obtained by blood testing.

### External Pressure Preconditioning

SUPERFICIAL BODY SURFACE PRECONDITIONING: As with ischemia induced by blockage of blood flow by compression over an artery such as by inflation of a blood pressure cuff, the induction of superficial pressure, to provide compression against an external body surface and thus restrict normal blood flow to the superficial tissues, can be implemented according to a schedule of transient induced pressure as required by any treatment or conditioning that may be expected. It is well known that cutaneous reactive hyperemia can be produced locally to occlude the microvessels on a skin surface by applying just enough pressure to induce visible redness upon release of the pressure. Greenwood et al., “Factors Affecting the Appearance and Persistence of Visible Cutaneous Reactive Hyperemia in Man,” 1: J Clin Invest. 1948 March; 27(2):187-97. Accordingly, the present inventors believe that ischemic conditioning can be provided by occluding the microvessels that are susceptible to superficial pressure and therefore empower the innate abilities of the conditioned superficial tissues for an anticipated intervention such as an incision or wound.

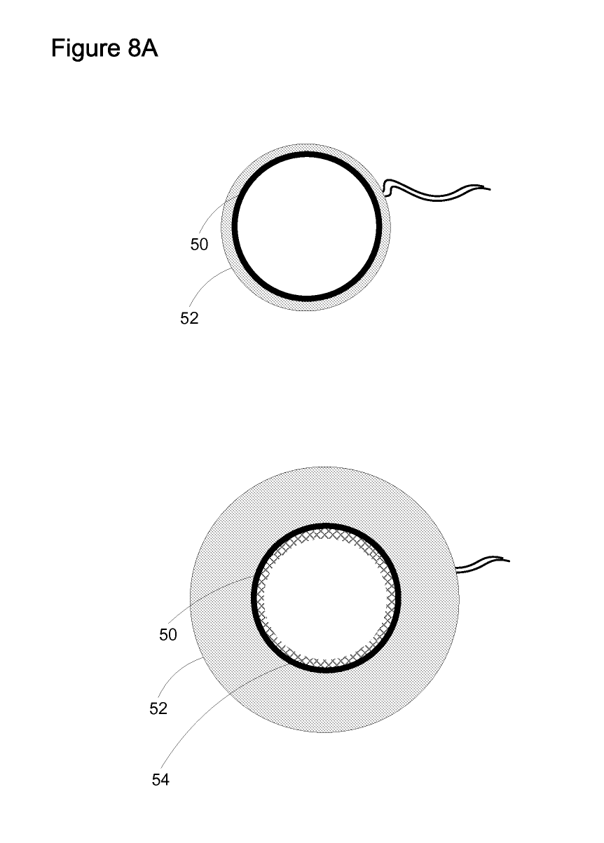

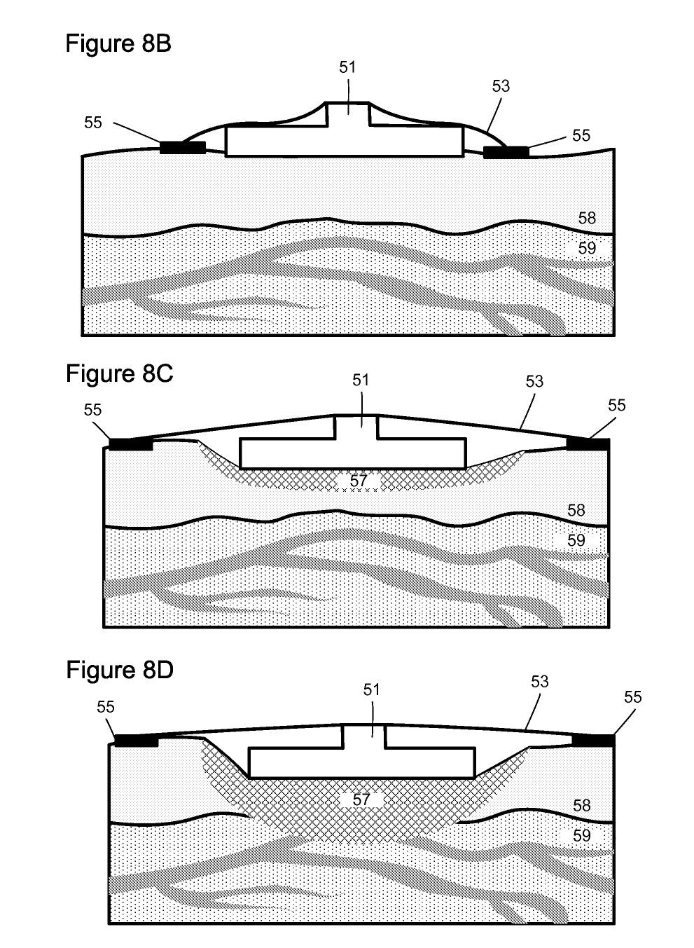

In one embodiment, the one or more administrations of superficial pressure can be provided as part of a design that includes, but is not limited to: a bed or chair, a tight-fitted garment, a pressured body suit, an adhesive wrap, an inflatable cuff, an expandable strap, or a weight, and combinations thereof. For example, FIG. 8A depicts cross sectional views of an embodiment of applying superficial pressure by an inflatable cuff (52) around an extremity (50). FIG. 8A depicts an embodiment of inflation of a cuff around an extremity to provide a small band of ischemia (54) beneath the surface of the extremity. In an embodiment, inflation of a balloon sectioned within another material such as a band that can be placed around the arm can provide localized superficial pressure around an extremity. Further, embodiments of weighted pressure and squeezing pressure can be adapted to provide pressure while being secured around an extremity.

In an embodiment, superficial pressure against a body surface such as the skin can be provided without completely wrapping around a part of the body. Such applications can be especially beneficial where proximal arterial supply is inaccessible or inconvenient, such as in applications for areas of the face, eyes, back, and chest among others. As depicted in the cross section views of FIGS. 8B-D, an occluding member (51) can be secured to a skin surface by an outer member (53) that has attaching members (55) capable of attaching to skin. As depicted, the outer member can be tightened by the attaching members to apply pressure to the occluding member. In an embodiment, the pressure applied to the occluding member can be manual, automated, combinations thereof, or any suitable in the art for the invention as described. In an embodiment, the outer member and attaching members can be part of a bandage and the occluding member can be a weight. In an embodiment, any method of applying superficial pressure can be used including but not limited to inflation, weighted pressure, and/or squeezing forces. In an embodiment, the ischemia (57) resulting from the superficial pressure can reach a dermal layer (58) alone as depicted in FIG. 8C, or also be capable of reaching subdermal layers (59) as depicted in FIG. 8D.

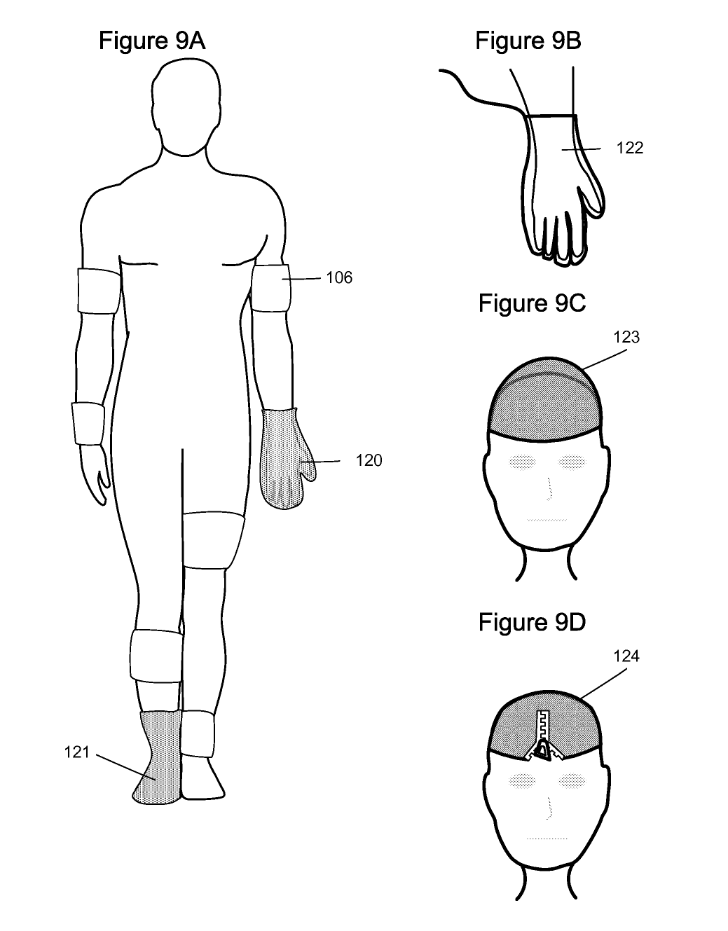

In one alternative embodiment as depicted in FIGS. 9A, 9B, 9C, and 9D, local ischemia of the superficial skin layers is provided by an inflatable mitten (120), inflatable sock (121), inflatable glove (122), inflatable cap (123), or zippered cap (124) that operates to provide compression against the skin and thus restrict normal blood flow to the superficial tissues. As with ischemia induced by blockage of blood flow by compression over an artery such as by inflation of a blood pressure cuff, the induction of superficial pressure can be implemented according to a schedule of transient induced pressure as pretreatment or preconditioning of areas that may be expected to be injured as a complication of a given medical or surgical intervention.

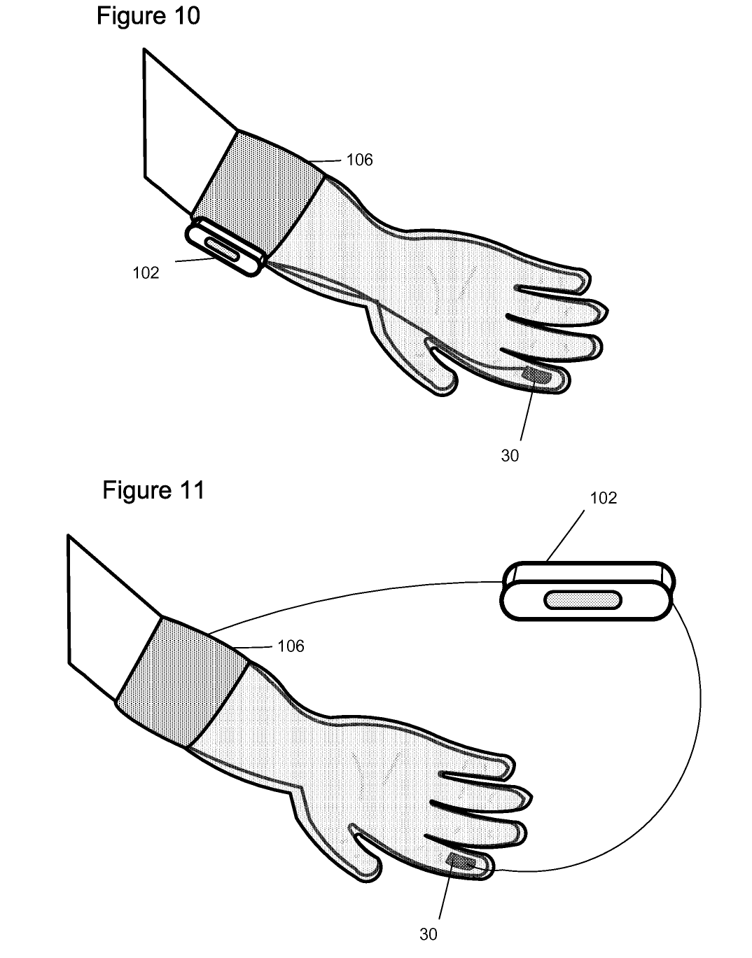

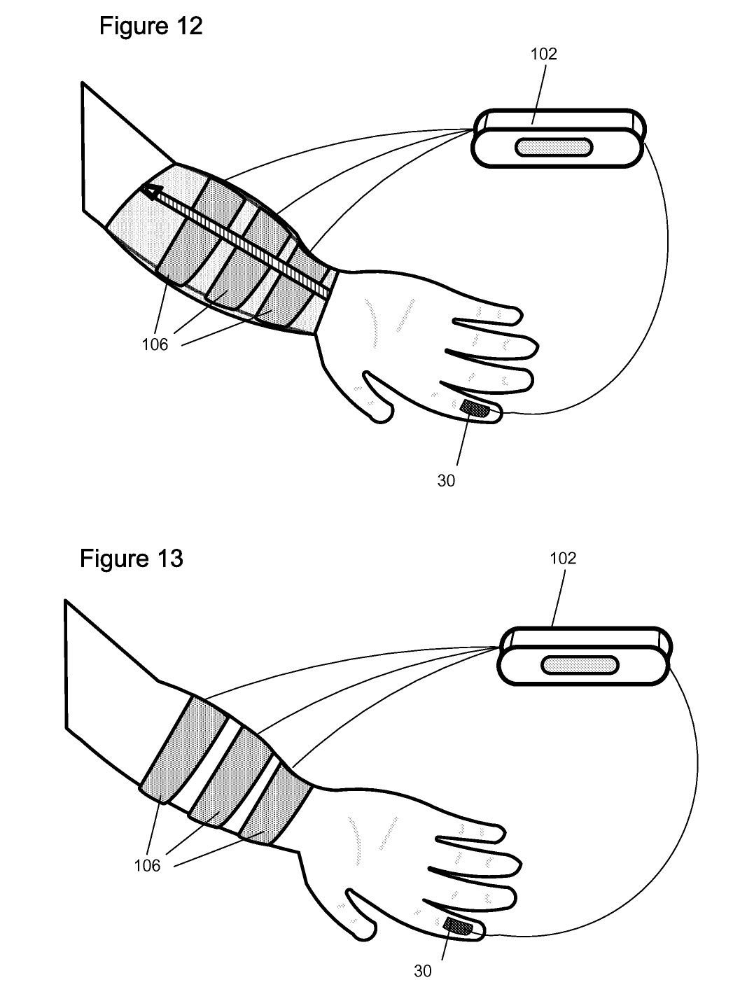

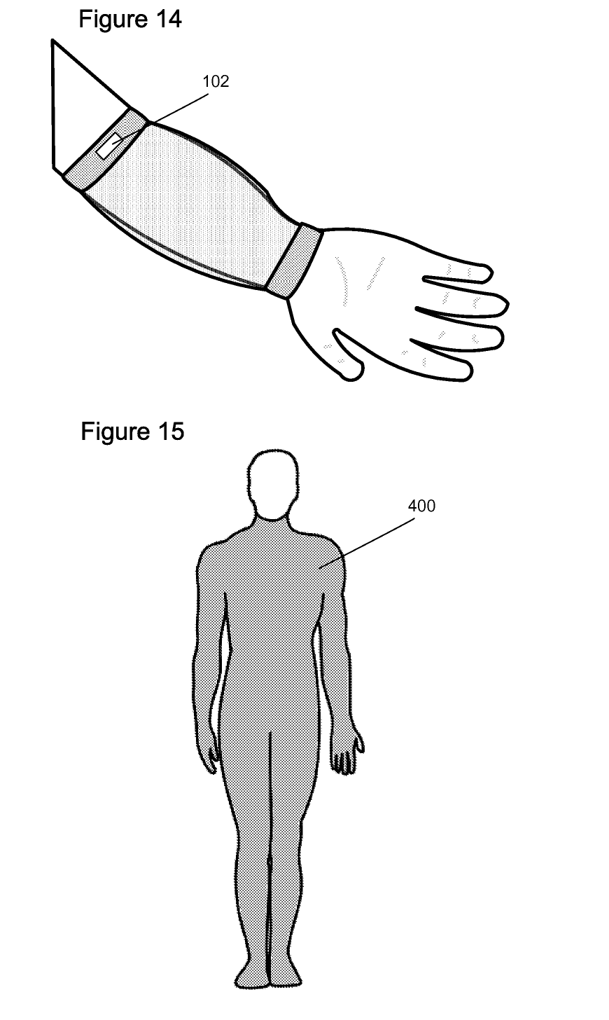

Several other embodiments for inflatable compression of the arm and hand are possible, as depicted by the illustrations of FIGS. 10-14. FIG. 10 depicts a glove adaptation with a sensor inside the glove and a controller (102) attached to the outside of the glove that controls the inflation of cuff (106). FIG. 11 depicts a glove adaptation with the sensor (30) also inside of the glove but the controller is unattached to the glove. FIG. 12 depicts a forearm glove adaptation secured to the arm with a zipper. Three cuffs (106) inside of the glove are provided to apply pressure when instructed by the unattached controller. A sensor (30) unattached to the glove is also provided for monitoring purposes. FIG. 13 depicts a forearm adaptation that is not gloved but has three cuffs and a sensor attached to a controller. FIG. 14 depicts a forearm glove adaptation that has the controller and/or monitoring integrated into a single glove device. Even further, in an embodiment, a full body suit can be used to provide ischemia to the superficial skin layers. FIG. 15 depicts an embodiment of a pressured body suit (400) that delivers external pressure to create ischemia at the skin and subdermal tissue levels.

In an embodiment, application of external superficial pressure can be provided for reduction of blood flow during the peak of blood flow during an intervention. For example, during chemotherapy, applying superficial pressure to reduce blood flow can reduce delivery of chemotherapy toxins to selected tissues. In an embodiment, applying superficial pressure to the head, e.g. via an inflatable or zippered cap, can reduce hair loss during chemotherapy by reducing the amount of toxins being delivered to hair follicles in the growth phase. In an embodiment, a cap for reducing hair loss can be adapted to fit a timer, zipper, inflation, or any other suitable apparatus to perform the invention as described herein. In an embodiment, the application of superficial pressure to reduce blood flow can be during a chemotherapy treatment. In an embodiment, applying superficial pressure during chemotherapy can be preceded by ischemic conditioning treatments before chemotherapy.

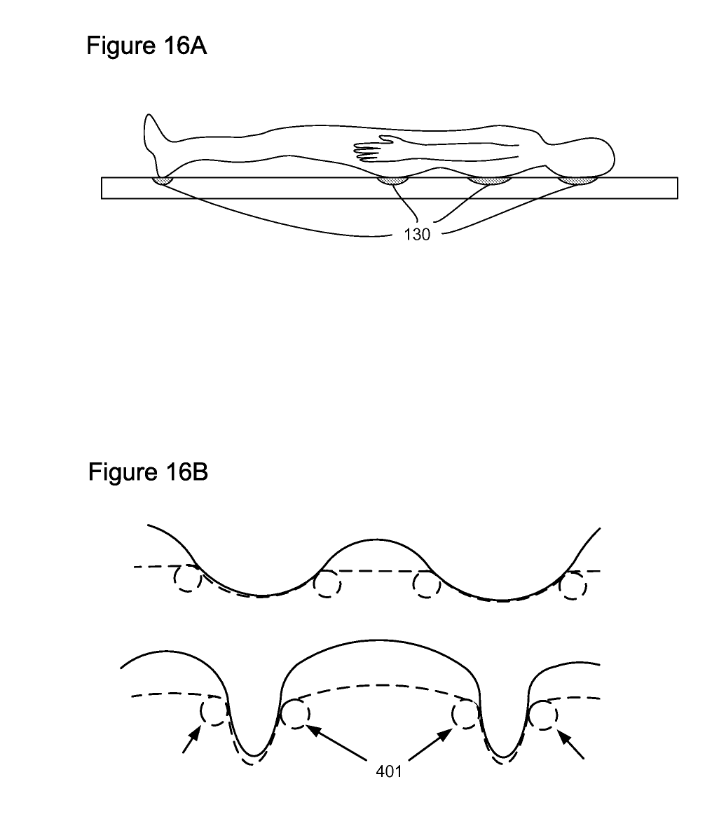

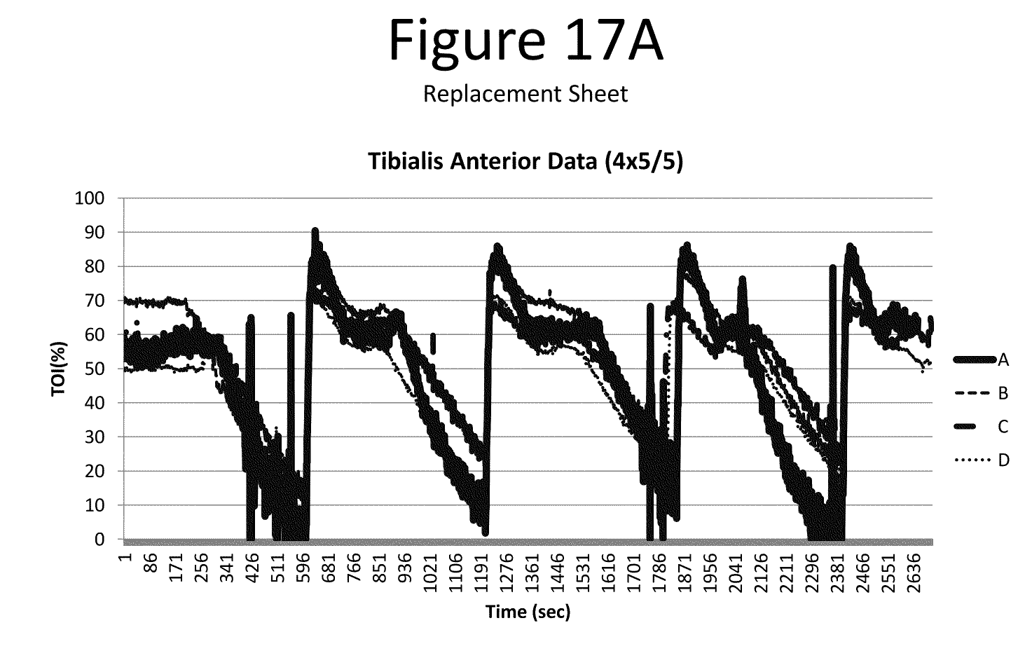

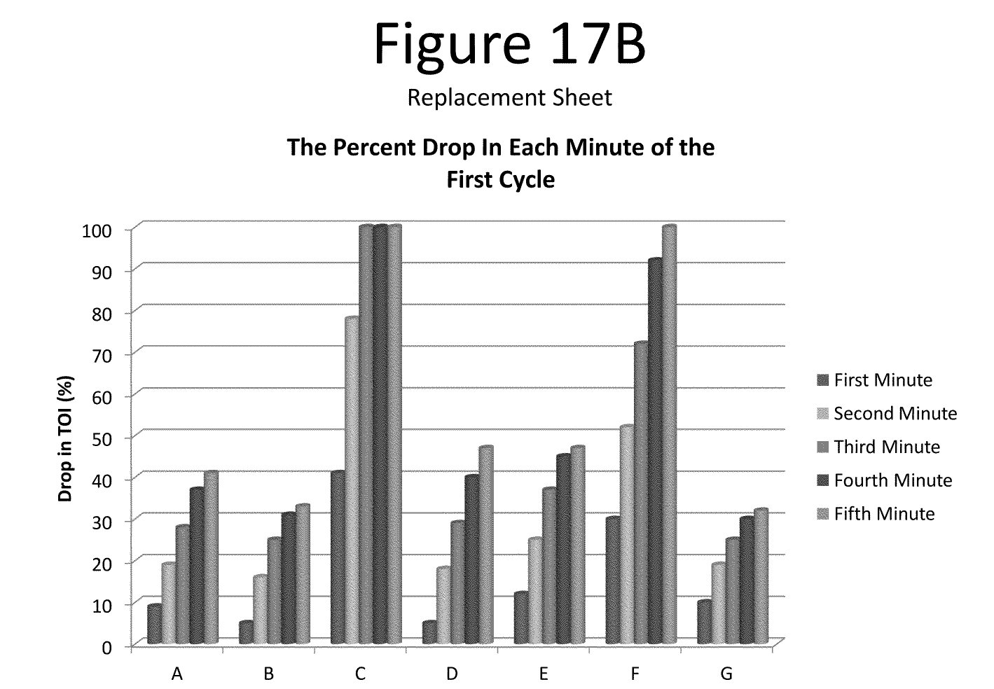

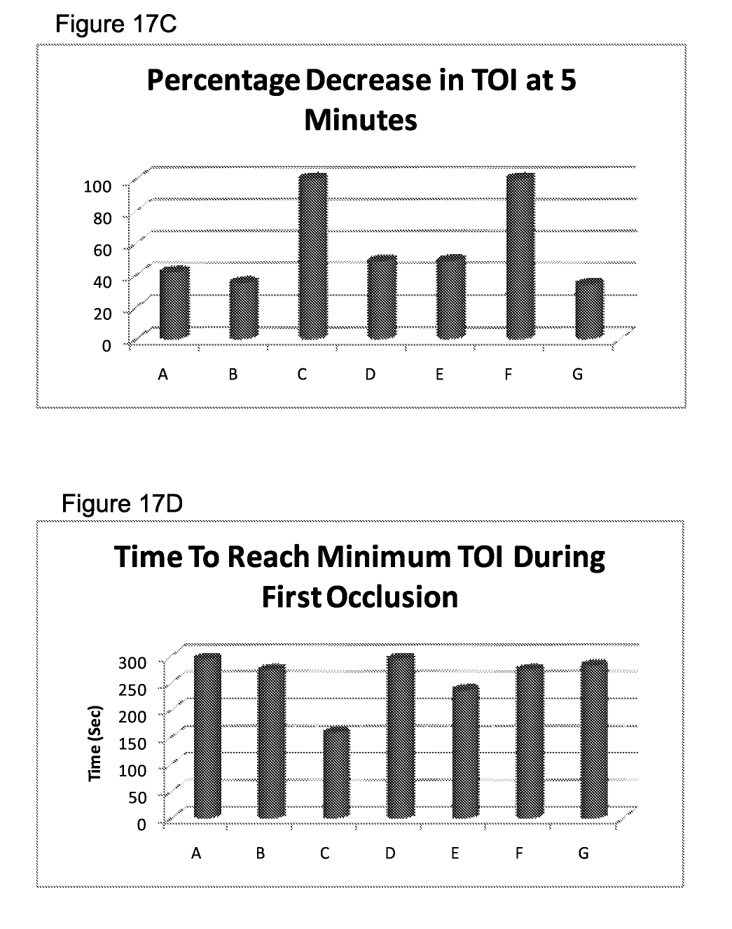

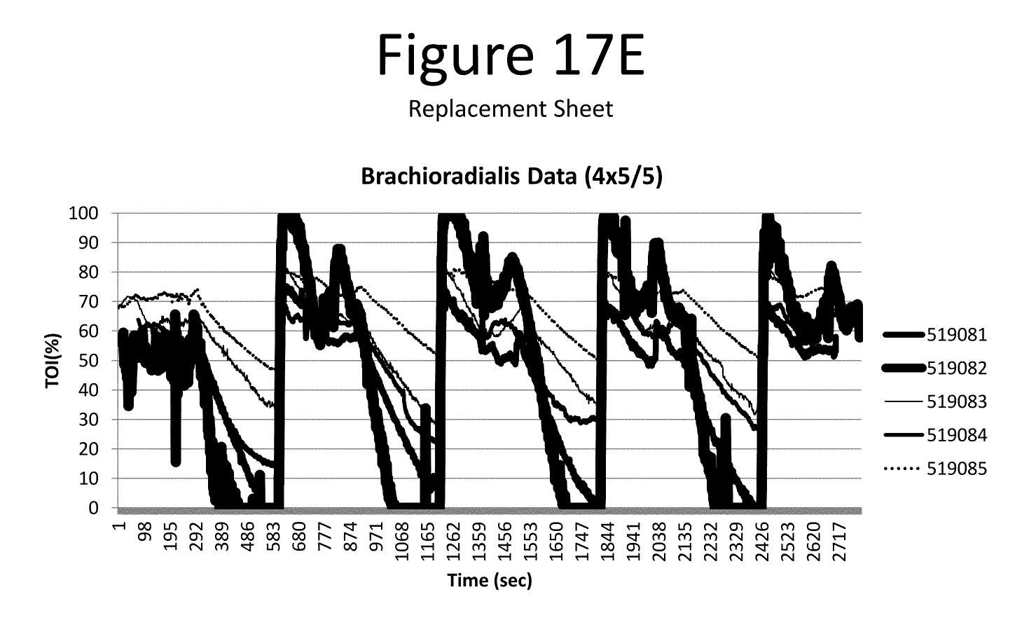

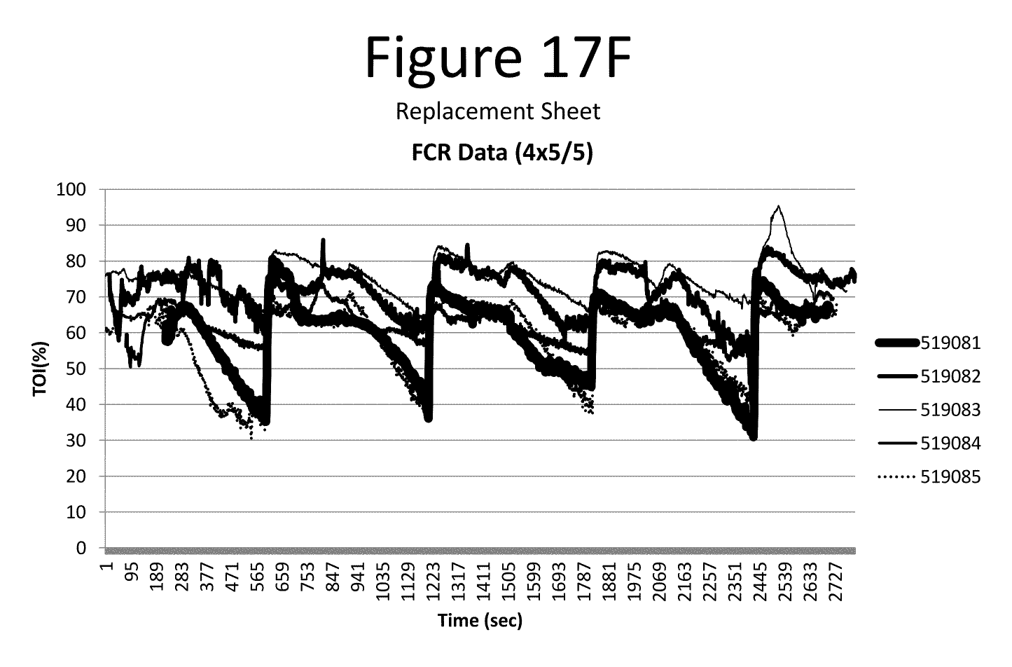

BEDSORES: In an embodiment, the invention as described herein can be particularly suited to apply superficial pressure for ischemic preconditioning of bedsores. As the skin dies, a bedsore starts as a red, painful area. Left untreated, the skin can break open and become infected. A sore can become deep, extending into the muscle, and is often very slow to heal. Pressure sores can develop on the buttocks, on the back of the head, the heels, the elbows, the hips, and/or any pressure point where the body contacts another surface. In an embodiment, a modified bed or mattress can be provided to apply superficial pressure to prevent or reduce bedsores. FIGS. 16A-B depict embodiments of a mattress capable of preventing or reducing bedsores by ischemic conditioning. In an embodiment when a patient is lying down on the mattress, the mattress can be capable of detecting pressure points (130) and treatment by an ischemic conditioning protocol using any suitable mechanism capable of applying superficial pressure, such as the skin squeezing mechanism depicted in FIG. 16B. As depicted in FIG. 16B, rollers or bars (401) are intermittently rolled together or tightened to provide transient ischemia and thus ischemic conditioning. Further, any suitable means for pressure detection or superficial pressure application that is well known in the art can be adapted for the present invention as described herein.

### Ischemic Conditioning to Improve Wound Healing

Wound healing is an important health care problem. Determining whether a wound is acute or chronic is the first step in understanding the components of healing or lack of healing Medical wounds can vary from being acute to chronic, or occurring following a repeated or persistent pattern. The acute care wound model of healing includes hemostasis, inflammation, proliferation, maturation, and is unique from chronic wound management. Chronic wounds are wounds that have failed to proceed through an orderly and timely process to produce an anatomic and functional integrity, or proceed through the repair process without establishing a sustained and functional result.

However, because each condition cannot be predicted and has variations for different patients, any ischemic conditioning therapy can be modified to suit the unique parameters for any particular condition. The present method of administering one or more transient ischemic episodes to the limb according to a schedule is neither dangerous nor expensive and may be readily implemented in every patient. The transient ischemic episodes provide protection and treatment against medical wounds by several mechanisms including without limitation: increased nitric oxide bioavailability, increased scavenging of free radicals and reduction in inflammation. If administered in a series of episodes over a sufficiently amount of time, the method is expected to increase arterial and smooth muscle flexibility, functional capillary density, and to hasten wound healing.

In an embodiment of the invention, the duration and frequency of ischemia targeted toward a tissue that is wounded or to be wounded may have a relationship with the effect of wound healing. Similar to perioperative outcomes, desired therapeutic effects within an early window and a delayed window of protection after conditioning are expected. Thus, in an embodiment, multiple separate ischemic conditioning treatments can be scheduled in any suitable manner as described herein, including but not limited to: several times daily, frequently over extended periods of time, based on assessments of specific interventions and/or treatment resistance, and combinations thereof. Further, in an embodiment, one or more of the ischemic conditionings directed towards acute wounds can be administered remotely from the targeted tissue that is wounded or to be wounded and provide a systemic effect. For example, occlusive cuffs can perform ischemic conditioning on an extremity, such as an arm or leg, to improve wound healing from an anticipated incision in a part of the body that is difficult to access for occlusion, like the back, chest, or torso.

In an embodiment of the invention, a scheduled series of transient ischemic episodes can be applied as conditioning to prevent or manage chronic wounds. Of the numerous compounds that are released following an ischemic episode as described herein, several may improve response to any wound or injury. For example, an increase in nitric oxide and adenosine bioavailability is known to occur after an ischemic episode. These compounds are frequently targeted by drug therapies and are well known to relax smooth muscle cells, decrease arterial stiffness, and improve wound healing over time. Accordingly, ischemic conditioning is able to noninvasively simulate ischemic effects of existing therapies. In an embodiment, ischemic conditioning can be administered supplemental to, or in addition to, conventional treatments of chronic wounds, such as heating, drugs, and irrigation.

For chronic wound treatment, separate ischemic conditioning treatments can also be scheduled in any suitable manner as described herein, including but not limited to: several times daily, frequently over extended periods of time, based on assessments of specific interventions and/or treatment resistance, and combinations thereof. Further, in an embodiment, one or more of the ischemic conditionings directed towards chronic wounds can be administered remotely from the targeted tissue that is wounded or to be wounded and provide a systemic effect. For example, occlusive cuffs can perform ischemic conditioning on an extremity, such as an arm or leg, to improve wound healing from an anticipated chronic wound.

In an embodiment, several tissue injuries resulting from chronic wounds can benefit from scheduled ischemic conditioning and the resulting increase in perfusion, relaxation of smooth muscle cells, vasodilation, anti-inflammatories, and anti-oxidants. For example, the vast majority of chronic wounds can be classified into three categories: venous ulcers, diabetic, and pressure ulcers. Venous ulcers, which usually occur in the legs, are thought to be due to venous hypertension caused by improper function of valves that exist in the veins to prevent blood from flowing backward. Ischemia often results from the dysfunction and, combined with reperfusion injury, causes the tissue damage that leads to the wounds.

Another major cause of chronic wounds, diabetes, is increasing in prevalence. Diabetics have a higher risk for amputation than the general population due to chronic ulcers. Diabetes also causes neuropathy, which inhibits the perception of pain. Thus patients may not initially notice small wounds to legs and feet, and may therefore fail to prevent infection or repeated injury, such as in the case for diabetic foot injuries. Further, diabetes causes immune compromise and damage to small blood vessels, preventing adequate oxygenation of tissue, which can cause chronic wounds. Pressure also plays a role in the formation of diabetic ulcers.

Other leading types of chronic wounds are pressure ulcers, which usually occur in people with conditions such as paralysis that inhibit movement of body parts that are commonly subjected to pressure such at the heels, shoulder blades, and sacrum. Pressure ulcers are caused by ischemia that occurs when pressure on the tissue is greater than the pressure in capillaries, and thus restricts blood flow into the area. For example, a bedsore develops when an area of the skin is under pressure and the blood supply to the skin is cut off for more than a few hours. Further, muscle tissue, which needs more oxygen and nutrients than skin does, shows some of the worst effects from prolonged pressure. Reperfusion injury damages tissue in pressure ulcers as in other chronic wounds.

In an embodiment, remote ischemic conditioning regimens for improving wound healing are performed at a hospital, medical clinic, or healthcare facility. In another embodiment, remote ischemic conditioning regimens for improving wound healing are performed at a subject's home.

### Ischemic Conditioning to Improve Rehabilitation

In an embodiment, repeated regimens of remote ischemic conditioning treatment are performed in a patient to improve the effects of rehabilitative therapies. One or more regimens may be performed in a single day, and regimens may be repeated 2, 3, 4, 5, 6, or 7 times a week. The beneficial effects of repeated ischemic conditioning treatments may be additive (stacking), or even multiplicative (synergistic). In a preferred embodiment, RIC treatments are performed on a subject's limb using an inflatable air cuff while one or more markers of tissue ischemia are being monitored. In a related embodiment, the RIC treatments are performed in an individual with significant cognitive and/or motor deficits which would otherwise prevent that individual from fully participating in intensive rehabilitative therapy sessions.

Remote ischemic conditioning elicits local (where the brief ischemic occlusions are performed) and systemic effects (in organs in tissues elsewhere in the body) which are anti-inflammatory, anti-apoptotic, pro-vascular, and pro-growth factor in nature. This environment is conducive to reparative processes that are occurring in the nervous system and neuromuscular systems. Thus, the benefits of performing one or more remote ischemic conditioning regimens in a physically impaired patient may manifest as improvements in cognition, motor, speech, special senses, gait, or any other ability dependent on nerve, neuromuscular junction, or muscle function.

In an embodiment, remote ischemic conditioning regimens for improving the effects of rehabilitative therapies are performed at a hospital, medical clinic, or healthcare facility. In another embodiment, remote ischemic conditioning regimens for improving the effects of rehabilitative therapies are performed at a subject's home.

What is claimed is:

1. A method for improving wound healing by ischemic conditioning treatments comprising:
a) measuring one or more baseline hemodynamic parameters of a subject;
b) applying an ischemic conditioning regimen on the subject comprising one or more ischemic conditioning treatments performed on one or more days; and
c) measuring post-ischemic parameters in the subject.

2. The method of claim 1, wherein the wound is anticipated to be caused by one or more conditions from the group consisting of: surgical operation, physical or chemical injuries, or pressure sores.

3. The method of claim 1, wherein ischemic conditioning is applied directly to the tissue subject to injury, or on tissue remote from an injury.

4. The method of claim 1, wherein the ischemic conditioning is performed between 72 to 24 hours before the anticipated injury, within 1 hour before the anticipated injury, or both.

5. The method of claim 1, wherein the ischemic conditioning is performed during the time period that a wound is created.

6. The method of claim 1, wherein the ischemic conditioning is performed between 30 seconds to 24 hours after a wound is created.

7. The method of claim 1, wherein ischemic conditioning treatments are performed periodically on multiple days over a time period from about 3 days to about 6 months.

8. The method of claim 1, wherein ischemic conditioning is performed at a hospital, medical clinic, healthcare facility, at a subject's home, or combinations thereof.

9. The method of claim 1, wherein ischemic conditioning is performed using a cuff-based system, one or more pressurizable garments, or combinations thereof.

10. The method of claim 1, wherein ischemic conditioning is performed directly on the tissue using a pressurizable bed mattress capable of applying localized pressure and causing ischemia.

11. The method of claim 1, combined with other methods of wound care.

12. A method for improving rehabilitative therapy by ischemic conditioning treatments comprising:
a) measuring one or more baseline hemodynamic parameters of a subject;
b) applying an ischemic conditioning regimen on the subject comprising one or more ischemic conditioning treatments performed on one or more days; and
c) measuring post-ischemic parameters in the subject.

13. The method of claim 12, wherein the subject has one or more deficits from the group consisting of: cognitive impairment, motor weakness, sensory impairment, and other physical or neurological impairments.

14. The method of claim 12, wherein ischemic conditioning is applied directly to an impaired limb or body area, or remote from the impaired limb or body area.

15. The method of claim 12, wherein ischemic conditioning treatments are performed periodically on multiple days over a time period from about 3 days to about 12 months.

16. The method of claim 12, wherein ischemic conditioning is performed at a hospital, medical clinic, healthcare facility, at a subject's home, or combinations thereof.

17. The method of claim 12, wherein ischemic conditioning comprises performing using a cuff-based system, one or more pressurizable garments, or combinations thereof.

18. The method of claim 12, wherein ischemic conditioning is performed on the tissue using a pressurizable bed apparatus capable of applying localized pressure and causing ischemia.

19. The method of claim 12, combined with other rehabilitative therapies.

20. A device for improving wound healing comprising a system for creating localized vascular occlusion and reperfusion; and a control device for controlling vascular occlusion and reperfusion in accordance with a schedule for ischemic conditioning treatments comprising a repeated combination of temporary vascular occlusion and reperfusion.

21. The device of claim 20, wherein the system for eliciting localized vascular occlusion comprises one or more elements from the group consisting of inflatable limb cuffs, pressurizable garments, and a pressurizable bed mattress capable of applying localized pressure to contacted skin and causing ischemia.

22. A device for improving rehabilitative therapy comprising a system for creating localized vascular occlusion and reperfusion; and a control device for controlling vascular occlusion and reperfusion in accordance with a schedule for ischemic conditioning treatments comprising a repeated combination of temporary vascular occlusion and reperfusion.

23. The device of claim 22, wherein the system for creating localized vascular occlusion comprises one or more elements from the group consisting of inflatable limb cuffs, pressurizable garments, and a pressurizable bed mattress capable of applying localized pressure to contacted skin and causing ischemia.
